(19) United States
(12) Patent Application Publication
(10) Pub. No.: US 2010/0150959 A1
(43) Pub. Date: Jun. 17, 2010

(54) PCV 2-Based Methods and Compositions for the Treatment of Pigs

(75) Inventors: Michael Sheppard, Eltham (AU); Sui T. Lay, Melbourne (AU)

Correspondence Address: MCANDREWS HELD & MALLOY, LTD, 500 WEST MADISON STREET, SUITE 3400, CHICAGO, IL, 60661, US

(73) Assignee: VECTOGEN PTY LTD., Perth (AU)

(21) Appl. No.: 12/621,607

(22) Filed: Nov. 19, 2009

Related U.S. Application Data
(60) Provisional application No. 61/122,555, filed on Dec. 15, 2008.

(51) Int. Cl. A61K 39/12; C12N 15/63; C07H 21/04; A61P 31/20

(52) U.S. Cl. 424/204.1; 435/320.1; 536/23.1

(57) ABSTRACT

The present invention relates to methods and compositions for vaccinating pigs against porcine circovirus associated diseases.

### RELATED APPLICATIONS

The present application claims priority of U.S. Provisional Patent Application No. 61/122,555, which was filed on Dec. 15, 2008. The entire text of the aforementioned application is incorporated herein by reference in its entirety.

### BACKGROUND OF THE INVENTION

Porcine circoviruses (PCV) are animal pathogens of the family circoviridae and are some of the smallest viruses replicating autonomously in mammalian cells. The virions are icosahedral, nonenveloped, 17 nm in diameter. Currently, there are two recognized types of PCV, porcine circovirus type 1 (PCV1) and porcine circovirus type 2 (PCV2). While PCV1 is nonpathogenic, PCV2 is associated with a variety of diseases and syndromes including but not limited to postweaning multisystemic wasting syndrome (PMWS), porcine dermatitis and nephropathy syndrome (PDNS), and congenital tremors collectively these may be referred to as porcine circovirus associated diseases (PCVAD). The diseases caused by PCV2 are now recognized to have a major economic impact in many pig-producing areas of the world.

Of particular commercial significance, PMWS can cause significant levels of mortality in many herds and severe economic losses to porcine industry. PMWS is a disease of nursery and fattening pigs characterized by growth retardation, paleness of the skin, dyspnea, and increased mortality rates. Initially identified in a swine herd in Canada in 1991, PMWS is now recognized as one of the most significant problems for the pig industry in the world. Various clinical studies have shown that PCV2 has etiological importance in PMWS.

PCV2 contains a single-stranded circular DNA genome of about 1.76 kb, having two major open reading frames (ORFs) (Mankertz et al., 2000). The capsid protein (Cap protein), encoded by ORF2 of the viral gene, is major structural protein of the virus and has type-specific epitopes (Mahe et al., 2000; Nawagitgul et al., 2000). Neutralizing monoclonal antibodies and neutralizing swine sera have been shown to react with the capsid protein (Pogranichnyy et al., 2000; McNeilly et al., 2001; Lekcharoensuk et al., 2004). An immuno-relevant ORF2 epitope of PCV2 has been identified as a serological marker for virus infection (Truong et al., 2001). Serologic analysis of PCV2 showed that the viruses could elicit hummoral immunity. The longer period of passive immunity is important for piglets to resist PCV2 infection and therefore less likely to show signs of PMWS (Blanchard et al., 2003a). It makes a PCV2 vaccine approach possible, if a vaccination method can be designed that will induce immunity in piglets prior to the time-point when weaning maternal immunity makes piglets susceptible to PCV2 infection. But there is no effective vaccine available.

The porcine adenovirus (PAdV) expression system is an attractive candidate for the production of a PCV2 vaccine. Porcine adenoviruses are able to replicate efficiently to high titers; provide cloning space; PAdV permit the expression of recombinant proteins in many porcine cell lines and tissues; express multiple genes in the same cell line or tissue; accurately express and modify the recombinant protein. Some studies have expressed the ORF2 protein of PCV2 by using the human adenovirus expression system and demonstrated the immunogenicity of the recombinant adenovirus in mice (Wang et al., 2006).

Nevertheless, while there have been several attempts to use the PCV2 ORF2 gene inserted into and expressed by a viral vector to elicit an appropriate protective immune response against PCVAD, such attempts have failed to produce a commercially feasible vaccine. It has been found that while ORF2 of PCV2 is a serological marker for associated diseases, when PCV2 ORF2 is expressed by a viral vector for vaccination purposes, such vaccines fail to produce a sufficiently appropriate immune response to protect pigs against disease. The present invention for the first time identifies a significant factor that leads to this failure and provides compositions that overcome the problems associated with the previous attempts to produce PCVAD vaccines based on viral vector delivered PCV2 ORF 2.

### SUMMARY OF THE INVENTION

The present invention addresses a need in the art for vaccines for treatment of pigs. In particular the inventors have discovered that in order to be effective in viral vector or subunit vaccine compositions, the PCV-2 ORF2 should be presented such that it is either secreted by the infected cell or is at least expressed on the cell surface of an infected cell.

In particular, the invention relates to a recombinant expression vector comprising a nucleic acid sequence that encodes a modified PCV2 ORF2 operably linked to a promoter, wherein the modified PCV2 ORF2 is one in which the nuclear localization signal of wild-type PCV2 ORF2 has been removed or modified to allow secretion of truncated ORF2 protein upon expression; or the modified PCV2 ORF2 is one in which the nuclear localization signal has been removed and replaced with a hydrophobic signal sequence that directs expression of the PCV2 ORF2 on the cell surface of an infected cell.

In specific embodiments, the recombinant expression vector is one in which the nuclear localization signal of the PCV2 ORF2 has been replaced with a hydrophobic signal sequence and cleavage site. The presence of the cleavage site will allow the expression product to be released as a secreted product. In specific embodiments, the nuclear localization signal of said ORF2 is replaced, for example, with the signal sequence selected from (but not limited to) the group consisting of chicken gamma interferon, porcine gamma interferon, and the HA protein of influenza virus. Many other signal sequences that may be used are described infra and also are to known to those skilled in the art.

The viral vector used may be any viral vector, including, for example, an adenoviral vector, an, adenoassociated viral vector, a lentiviral vector, a herpes viral vector, a pox viral vector. In particular, the viral vector is a porcine viral vector. In more specific embodiments, the adenoviral vector is a porcine adenoviral vector selected from the group consisting of PAdV1, PAdV2, PAdV3, PAdV4, and PAdV5. In certain preferred embodiments, the porcine adenoviral vector is PAdV3. It is preferable that the PAVd3 is a replication competent PAdV3. In other embodiments, the nucleic acid sequence that encodes said modified PCV ORF2 is inserted into a non-essential sequence in PAdV3.

Exemplary non-essential sequence of PAdV-3 is selected from the group consisting of the E3 region, ORF 1-2 and 4-7 of E4, the region between the end of E4 and the ITR of the porcine adenovirus genome.

In other embodiments, the PAdV3 is a recombinant PAdV3 comprising a fibre gene native to said PAdV3 and further comprising a second fibre gene that is heterologous to said adenovirus, wherein said second fibre gene is acquired by said recombinant adenovirus by growth of said recombinant adenovirus in a cell line that stably expresses said second fibre gene. In preferred embodiments, the nucleic acid comprises the sequence of SEQ ID NO:1, SEQ ID NO:3 or SEQ ID NO:5.

In still other preferred embodiments, the recombinant expression vector further comprises a nucleic acid that encodes another antigen for eliciting an immune response in pigs. For example, such an additional antigen may be selected from the group consisting of the additional antigen of another porcine pathogen is selected from the group consisting of an antigen of PRRS virus, an antigen of Mycoplasma hypopneumoniae, an antigen of Actinobacillus pleuropneumoniae, an antigen of E. coli, an antigen of Atrophic Rhinitis, an antigen of Pseudorabies virus, an antigen or Hog cholera, an antigen of Swine Influenza, and combinations thereof. Preferably, the antigen is from the group consisting of an antigen of PRRS virus, an antigen of atrophic rhinitis, an antigen of Pseudorabies virus, an antigen or Hog cholera, an antigen of Swine Influenza, and combinations thereof.

The invention contemplates a composition comprising a first recombinant expression vector as described above and a second recombinant expression vector that comprises an additional antigen for eliciting an immune response in pigs. Also contemplated are vaccines for eliciting a protective response against PCV2 infection in pigs comprising such a composition.

Other aspects of the invention relate to a vaccine for eliciting a protective response against porcine circovirus (PCV2) infection in pigs comprising a veterinarily acceptable vehicle or excipient and a recombinant expression vector comprising a nucleic acid sequence that encodes a modified PCV2 ORF2 operably linked to a promoter, wherein the modified PCV2 ORF2 is one in which the nuclear localization signal of wild-type PCV2 ORF2 has been removed or modified to allow secretion of truncated ORF2 protein upon expression; or the modified PCV2 ORF2 is one in which the nuclear localization signal has been removed and replaced with a signal hydrophobic signal that directs expression of the PCV2 ORF2 on the cell surface of an infected cell. In some embodiments, the vaccine may advantageously further comprise one or more additional antigen for vaccination of pigs wherein said additional one or more antigen is provided as a protein component in the veterinarily acceptable vehicle or excipient of said vaccine.

The invention specifically contemplates preparation and use of a vaccine for the protection of pigs against diseases caused by PCV-2 ORF2, said vaccine comprising a recombinant virus vector comprising a promoter operably linked to a hydrophobic signal sequence comprising a nucleic acid that encodes a membrane anchoring domain, a multiple cloning site for insertion of a modified PCV-2 ORF2 in frame with said hydrophobic signal sequence, a polyadenylation signal; and a viral genome, wherein said modified PCV-2 ORF2 lacks a nuclear localization signal. In specific embodiments, the vector further comprises a cleavage sequence immediately upstream of the cloning site for modified PCV-2 ORF2, wherein the PCV-2 ORF 2 expression product from said vector produces a soluble gene product.

Also contemplated is preparation and use of a vaccine for the protection of pigs from PCV-2 associated disorder, said vaccine comprising a recombinant porcine adenovirus 3 vector comprising a promoter operably linked to a hydrophobic signal sequence comprising a nucleic acid that encodes a membrane anchoring domain, and a nucleic acid that encodes a truncated PCV2 ORF2 that lacks a NLS sequence inserted in frame with said hydrophobic signal sequence, a polyadenylation signal; and a porcine adenovirus 3 genome.

The vaccines may be formulated for any route of administration including for example oral, nasal, intramuscular, subcutaneous, or intradermal delivery. In preferred embodiments, the vaccine is formulated for aerosol administration.

The invention also contemplates a method for eliciting an immune response in a porcine subject comprising administering vaccines of the invention to the porcine subject in an amount effective to elicit a protective immune response in said porcine subject.

In specific embodiments, the methods reduce viral load of porcine circovirus 2 (PCV2) in a pig comprising inducing an immunological or immunogenic response against PCV2 in the pig comprising administering to the pig a composition comprising a pharmaceutically or veterinarily or medically acceptable carrier and an expression vector comprising a nucleic acid sequence that encodes a modified PCV2 ORF2 operably linked to a promoter, wherein the modified PCV2 ORF2 is one in which the nuclear localization signal of wild-type PCV2 ORF2 has been removed or modified to allow secretion of truncated ORF2 protein upon expression; or the modified PCV2 ORF2 is one in which the nuclear localization signal has been removed and replaced with a signal hydrophobic signal that directs expression of the PCV2 ORF2 on the cell surface of an infected cell.

In specific embodiments, the administering is performed prior to breeding. In still other embodiments, the pig that is administered the vaccine is a pregnant female pig.

### BRIEF DESCRIPTION OF SEVERAL VIEWS OF THE DRAWINGS

FIG. 1. Schematic for preparation of recombinant vectors of the invention.

FIG. 2. A collection of eukaryotic signal sequences reproduced from FIG. 1 of Heijne Eur. J. Biochem 133, 17-21 (1983). The sequences are aligned based on their known or predicted cleavage sites, which are indicated by an asterisk (*).

FIG. 3. PCV2 Vaccination/Challenge Trial: Percentage virus isolation from piglets post challenge in each of groups treated with (1) PAdV3-PCV2ORF2 full length; (2) PAdV3-PCV2ORF2 truncated; (3) PAdV3-PCV2ORF2 secreted; and (4) with phosphate buffered saline (control).

FIG. 4. PCV2 Vaccination/Challenge Trial: Number of days post challenge with all pigs (in a group) free of any adverse clinical signs in each of groups treated with (1) PAdV3-PCV2ORF2 full length; (2) PAdV3-PCV2ORF2 truncated; (3) PAdV3-PCV2ORF2 secreted; and (4) with phosphate buffered saline (control).

### DESCRIPTION OF ILLUSTRATIVE EMBODIMENTS

PCVAD are serious diseases that causes significant economic harm in the pig-farming industry. While the etiological marker of this disease has been identified as PCV2 ORF2, all attempts thus far to produce a viral vector PCV2-ORF2-based vaccine against these diseases have failed to produce a commercially significant vaccine. The present invention for the first time provides viral vaccine compositions that comprise a modified PCV ORF2 that provides immunity against PCV2.

The full length nucleic acid sequence of PCV2 ORF2 has previously been characterized and is shown in SEQ ID NO:7. This nucleic acid encodes a protein of SEQ ID NO:8. The first 42 codons of SEQ ID NO:7 (shown in SEQ ID NO:9) encode a nuclear localization signal for PCV ORF2 (Liu et al., Virology 285: 91-99, 2001). In nuclear targeting studies, Liu et al. prepared PCV2 ORF2 fusion proteins with green fluorescent protein and showed that when the signal at amino acid residues 1 to 41 of PCV2 ORF2 is removed, the PCV ORF2 GFP fusion protein became cytoplasmic. Liu et al. thus concluded that residues 1 to 42 and in particular, basic residues at positions 12 to 18 and 34 to 41 were essential to the nuclear localization of PCV2 ORF2.

The present inventor has found that removing the native nuclear localization sequence (i.e., the sequence at residues 1 to 42 of SEQ ID NO:8) and replacing it with a signal sequence that causes secretion of the PCV2 ORF2 from the cell renders a composition containing such a modified PCV2 ORF2 encoding nucleic acid useful as a viral vectored vaccine for producing immunity against PCVAD. The following discussion provides methods and compositions for making and using such vaccines and for treating pig populations with such vaccines.

The present invention relies on conventional techniques for the construction of improved viral vaccines for the treatment of pigs. The viral vaccines may be constructed from any viral vector that can be used to infect pigs and may include vectors such as but not limited to an adenoviral vector, an adenoassociated viral vector, a lentiviral vector, a herpes viral vector, a pox viral vectors. In exemplary embodiments, the viral vectors are porcine adenoviral vectors. Vaccines made with porcine viral vectors are known to those of skill in the art (see e.g., U.S. Pat. Nos. 7,323,177; 7,297,537; 6,852,705).

The present invention relates to methods of preparing and use of recombinant viral vaccine compositions that can be administered to a population of pigs for protective immunity against any diseases caused by PCV-2. Advantageously, the vaccine constructs of the invention direct expression of the PCV-2 ORF2 antigen being delivered to an extracellular site on the infected cell rather than internal expression of the PCV-2 ORF2. In the case of the vaccines described herein, the immunogen is thus delivered to the outer surface of mucosal cells (e.g., mucosal cells in the nasal passages, the respiratory tract, the gastrointestinal tract, the intestinal mucosa and the like) thereby presenting the immunogen at a site where an immune response may rapidly be mounted as opposed to expression of the delivered PCV-2 ORF2 immunogen within the cells where it may not come into efficient contact with the appropriate immune response machinery.

The existing vaccines do not meet the long-felt need in the art for an effective vaccine against diseases caused by PCV-2. To combat the problems with the existing treatments PCV-2 related diseases, the present inventors have developed a new vaccine for conferring protective immunity to pigs. The vaccine is based on a viral expression system (any virus that infects pigs may be used as the delivery virus) e.g., a porcine adenovirus expression system that affords expression of a modified form of PCV-2 ORF in a subunit vaccine. The antigen is expressed in-frame with a hydrophobic signal sequence and is either presented on the cell surface of virus-infected cells in the pig to which the vaccine has been administered or is alternatively secreted into the extracellular domain in such infected animal in the event that the expression vector is one in which the hydrophobic signal sequence also comprises a cleavage signal. These features and methods and compositions for using recombinant viral vaccines for PCV-2 related diseases are described in further detail herein below.

In general terms the vaccine of the present invention is comprised of a viral expression vector that is made of a viral genome. Porcine adenoviruses are well known to those of skill in the art and have been extensively characterized. In specific embodiments, the porcine adenovirus 3 is used as the vector in the methods and compositions described herein. Given the teaching provided herein however, the skilled person may use any virus that infects pigs to prepare vaccines of the invention.

In the vaccines prepared herein the promoter used may be any promoter that can drive expression of a heterologous gene of interest in an viral construct. Such promoters include but are not limited to avian adenoviral major late promoter (MLP), CMVp, PGK-, E1-, SV40 early promoter (SVG2), SV40 late promoter, SV-40 immediate early promoter, T4 late promoter, and HSV-1 TK (herpesvirus type 1 thymidine kinase) gene promoter, the RSV (Rous Sarcoma Virus) LTR (long terminal repeat) and the PGK (phosphoglycerate kinase) gene promoter. Many other mammalian or avian promoters known to those of skill in the art also may be used.

The promoter used in the vaccines described herein drives the expression of an in-frame fusion of a hydrophobic signal sequence linked in-frame with a PCV-2 ORF 2-encoding nucleic acid sequence. The hydrophobic signal sequence may be any sequence that can be used to target or specifically direct the expression of the nucleic acid of interest to the outer membrane of the host cell that is infected with the expression vector. In the present invention the PAV-based expression vector is intended to infect pigs. The FAV typically infects mucosal cells, liver and epithelial cells which may be found for example in the intestinal tract, the respiratory tract or the gastrointestinal tract of the animal. Thus, the hydrophobic signal sequence is one which traffics the expression of the PCV-2 ORF2 expression product on the cell surfaces of these mucosal cells. By thus presenting the PCV-2 ORF2 expression product at the cell surface of mucosal cells in the animal, the vaccine of the invention are able to most effectively deliver the antigen to the internal site where an immune response can be effectively mounted as opposed to expression within the cell of animal where it may be less effective at facilitating the mounting of an immune response.

In eukaryotic cells, secretory proteins are targeted to the endoplasmic reticulum membrane by hydrophobic signal sequences. The present invention uses this property to employ heterologous hydrophobic signal sequences to direct the expression of a given protein in the vaccine to the cell surface.

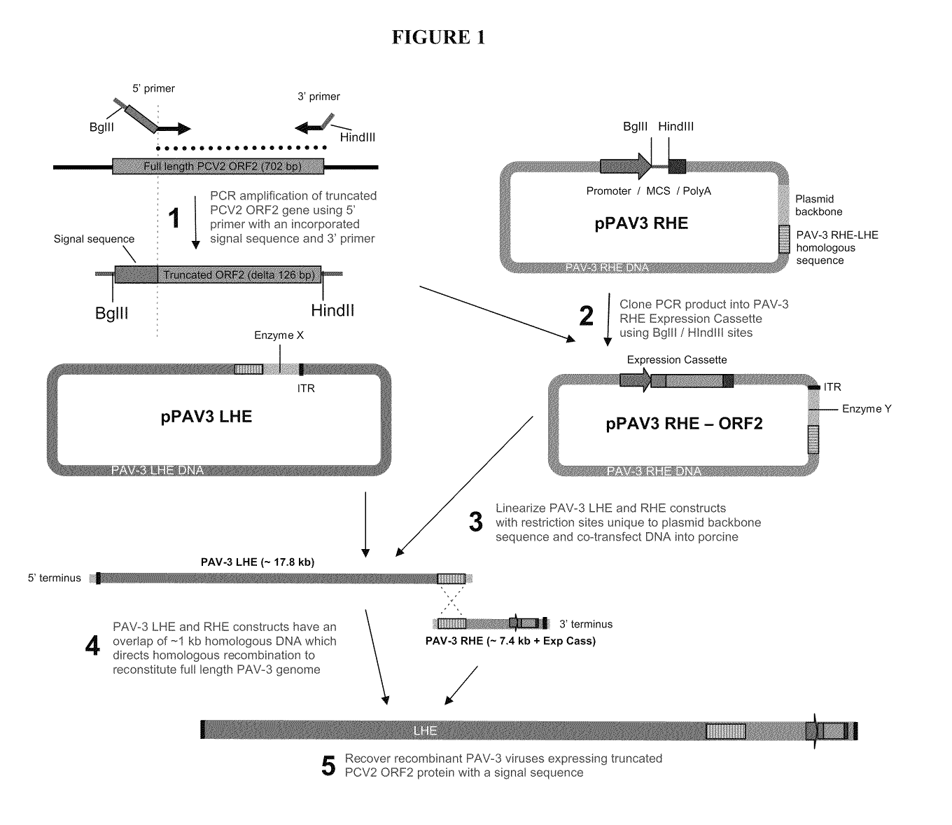

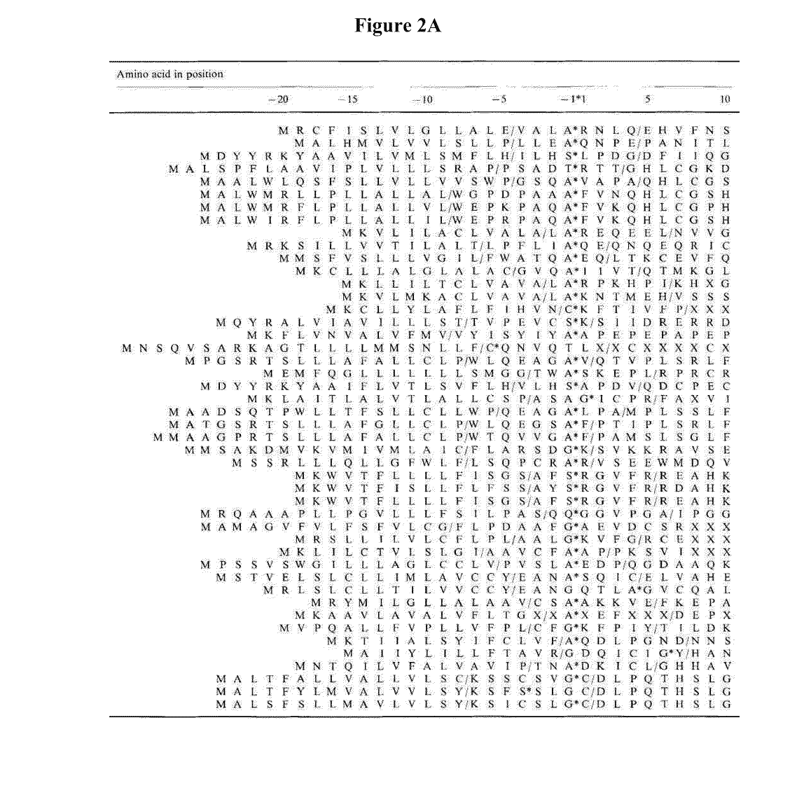

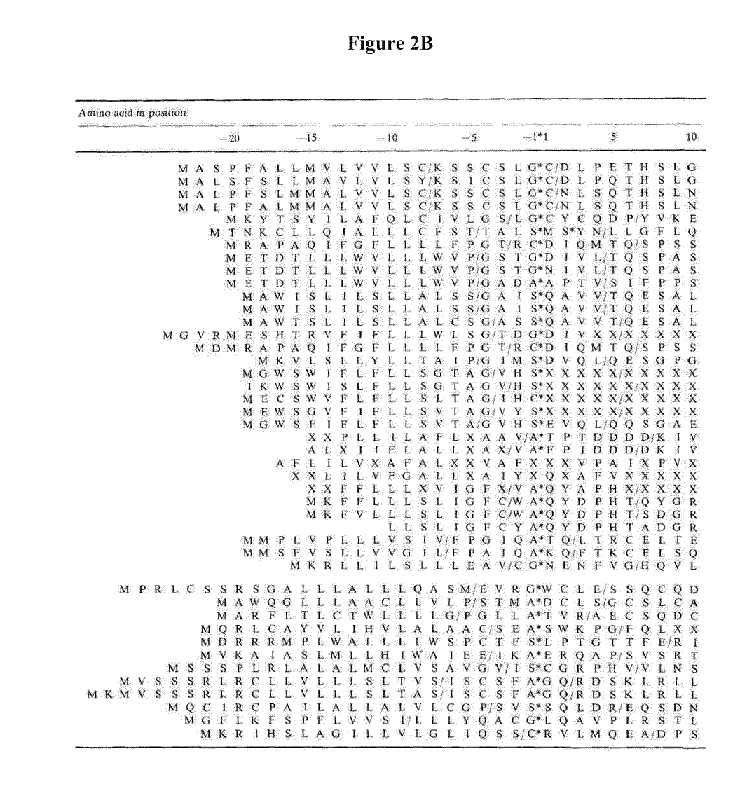

The viral vectors employed herein are recombinant vectors in that they comprise a polynucleotide construct that contains nucleic acid that encodes a modified PCV2 ORF2 in which the native nuclear localization signal of wild-type PCV2 ORF2 has been removed and replaced with a signal sequence and cleavage site to allow secretion (from the infected cell) of truncated ORF2 protein upon expression. For example, the native nuclear localization sequence (NLS) of ORF2 could be replaced with the signal sequence from chicken gamma interferon, porcine gamma interferon, or the HA protein of influenza virus. Other signal sequences that may be used include, for example, the signal sequence of whey phosphoprotein signal sequence; α-1 acid glycoprotein; α-thyrotropin; insulin from hagfish; insulin from anglerfish; human insulin; rat insulin I or II; ovine β-casein; ovine x-casein; ovine α-lactalbumin; ovine β-lactoglobulin; ovine α-sl casein, and ovine α-s2 casein; VS virus glycoprotein; cockerel VLDL-11; bee melittin; rat lactin; human placental lactogen; human β-choriogonadotropin; human α-choriogonadotropin; rabbit uteroglobin; rat growth hormone; human growth hormone; bovine growth hormone; bovine parathyroid hormone; rat relaxin; rat serum albumin; human serum albumin; rat liver albumin; chicken tropoelastin B; chicken ovomucoid; chicken lysozyme; chicken conalbumin; human α-1 antitrypsin; rat prostatic binding protein; rat prostatic binding protein c2; AD virus glycoprotein; rat apolipoprotein A1; rabies virus glycoprotein; human influenza Victoria hemagglutinin; human influenza Jap hemagglutinin; avian influenza FPV hemagglutinin; human leukocyte interferon; human immune interferon; human fibroblast interferon; mouse χ-immunoglobulin; mouse λ-immunoglobulin; mouse x-immunoglobulin; mouse H-chain immunoglobulin; mouse embryonic VH-immunoglobulin; mouse H-chain immunoglobulin; canine trypsinogen 1; canine trypsinogen 2+3; canine chymotrypsinogen 2; canine carboxypeptidase A1; canine amylase; mouse amylase; rat amylase; rabbit α-lactalbumin; porcine α-lactalbumin; rat carboxypeptidase A; bovine ACTH-β-LPH precursor; porcine ACTH-β-LPH precursor; human ACTH-β-LPH precursor; porcine gastrin; mouse renin; trypanosome glycoprotein; catfish somatostatin; anglerfish somatostatin; rat calcitonin; and anglerfish glucagons. Each of these signal sequences is shown at FIG. 1 of von Heijne et al. Eur. J. Biochem 133 17-21 (1983) and may readily be adapted for use herein. The signal sequences from FIG. 1 of the aforementioned reference are reproduced in FIG. 2 herein.

These and other signal peptide sites for a given protein can readily be determined using methods known to those of skill in the art. For example, signal peptide site can be predicted using the SignalP 3.0 server (Bendtsen, J. D., Nielsen, H., von Heijne, G. & Brunak, S. (2004) Improved prediction of signal peptides: SignalP 3.0. J. Mol. Biol. 340, 783-795). Additionally, there are websites available to facilitate determination of signal sequences see e.g., http://www.cbs.dtu.dk/services/SignaP/. The exact identity of the signal sequence used is not important as long as it is a hydrophobic sequence that is capable of trafficking the expressed product to the cell surface.

In preferred embodiments, the signal sequence contains a cleavage site that permits the signal sequence to be cleaved and allows the attached protein to be secreted to the extracellular space of such cells. In particularly preferred embodiments, this aspect of the invention is demonstrated using the signal sequences from chicken gamma IFN which contains sequence: MTCQTYNLFVLSVIMIYYGHTASSLNL (SEQ ID NO:12) encoded by the DNA sequence of ATG ACT TGC CAG ACT TAC AAC TTG TTT GTT CTG TCT GTC ATC ATG ATT TAT TAT GGA CAT ACT GCA AGT AGT CTA AAT CTT (SEQ ID NO:11), a hydrophobic signal sequence for porcine gamma IFN is: MSYTTYFLAFQLCVTLCFSGSYC (SEQ ID NO:14), which is encoded by the DNA sequence of ATG AGT TAT ACA ACT TAT TTC TTA GCT TTT CAG CTT TGC GTG ACT TTG TGT TTT TCT GGC TCT TAC TGC (SEQ ID NO:13), a hydrophobic signal sequence for human influenza virus H1N2 is: MKVKLLILLCTFTATYADTI (SEQ ID NO:16) encoded by a sequence of atg aaa gta aaa cta ctg atc ctg tta tgt aca ttt aca get aca tat gca gac aca ata (SEQ ID NO:15). Each of these exemplary sequences also contain a cleavage site at which a signal peptidase acts and results in the release of the expressed gene product of the gene of interest. The putative cleavage sites of the sequence from FIG. 2 are marked with an asterisk (*).

The polynucleotide construct will preferably comprise DNA that encodes the protein to be delivered. Such DNA may be comprised of the nucleotide bases A, T, C, and G, but also may include any analogs or modified forms of such bases. Such analogs and modified bases are well known to those of skill in the art, and include but are not limited to methylated nucleotides, internucleotide modifications such as uncharged linkages and thioates, use of sugar analogs, and modified and/or alternative backbone structures, such as polyamides.

In exemplary embodiments, the viral vectors are porcine adenovirus vectors. The porcine adenovirus vectors may be replication-competent or replication-defective in a target cell. In the event that the vectors are replication-defective, the vectors may require use of a helper cell or a helper virus to facilitate replication. Use of helper cells or helper viruses to promote replication of replication-defective adenoviral vectors is routine and well-known in the art. Typically, such helper cells provide the function of the entity that has been knocked out of the recombinant adenoviral vector to render it replication defective.

A replication competent vector on the other hand may be referred to as a “helper-free virus vector” in that it does not require a second virus or a cell line to supply something defective in the vector. As noted above, modification of the PCV2 ORF2 to remove the NLS and the addition of a signal sequence with cleavage site converts the ORF2 protein from being one that is localized in the nucleus to being one that is secreted from the cell. The secretion of the to expression product from the cell into the extracellular space renders the vaccine containing the modified PCV2 ORF2 more effective in stimulating antibody production than a vaccine that expresses a PCV2 ORF2 that contains the NLS. This extracellular secretion of the ORF2 expression product is an advantage over the previously described vaccines as it leads to a greater antibody immune response than is seen when the vaccine is prepared with PCV2 ORF2 having a wild-type NLS.

The preparation of viral vector-based vaccines that contain the modified PCV2 ORF2 is limited only by the insertion capacity of the given viral genome and ability of the recombinant viral vector to express the inserted heterologous sequences. For example, where the vector is an adenoviral vector, adenovirus genomes can accept inserts that increase the size of the recombinant adenovirus to at least 105% of the wild-type genome length and remain capable of being packaged into virus particles. The insertion capacity of such viral vectors can be increased by deletion of non-essential regions and/or deletion of essential regions, such as, for example, E1 function, whose function can then be provided by a helper cell line, such as one providing E1 function. In some embodiments, a heterologous polynucleotide encoding the protein of interest (in this case the PCV2 ORF2 and/or any additional therapeutic protein that is to be used in the vaccine) is inserted into an adenovirus E3 gene region. In other embodiments, the non-essential portions of the E3 region are deleted and the heterologous polynucleotide encoding the protein(s) of interest is inserted at that gap left by the deletion. In some preferred embodiments, where the recombinant adenoviral vector is a porcine adenovirus serotype 3 (PAdV-3) based adenoviral vector, in which the expression construct containing the PCV2 ORF2 encoding nucleic acid (and/or other nucleic acid) is inserted into the region of the PAdV-3 genome located after the polyadenylation signal for PAdV-3 E3 and before the start of the ORF for the PAdV-3 fibre gene.

In some embodiments, an adenovirus is created where the insertion or the deletion followed by the insertion is in the E1 gene region of the adenovirus the vector is then propagated in a helper cell line providing E1 function. Other regions of PAdV-3 into which the heterologous gene may be inserted include the E4 region. Where the recombinant adenoviral vector is a PAdV-3 based vector, the entire E4 region, except that region that encodes ORF3 can be deleted to make room for the heterologous gene. For example, the region at map units 97-99.5 is a particularly useful site for insertion of the heterologous gene. As shown in Li et al. (Virus Research 104 181-190 (2004)), the PAdV-3 E4 region located at the right-hand end of the genome is transcribed in a leftward direction and has the potential to encode seven (p1-p7) ORFs. Of these only ORF p3 is essential for the replication. As such, much if not all of the rest of the E4 region may readily be deleted without rendering the virus replication defective, thereby allowing for more room for heterologous inserts. In one embodiment of the invention, insertion can be achieved by constructing a plasmid containing the region of the adenoviral genome into which insertion of the polynucleotide encoding for a desired therapeutic protein is desired. The plasmid is then digested with a restriction enzyme having a recognition sequence in that adenoviral portion of the plasmid, and a heterologous polynucleotide sequence is inserted at the site of restriction digestion. The plasmid, containing a portion of the adenoviral genome with an inserted heterologous sequence, is co-transformed, along with an adenoviral genome or a linearized plasmid containing the adenoviral genome into a bacterial cell (such as, for example, E. coli). Homologous recombination between the plasmids generates a recombinant adenoviral genome containing inserted heterologous sequences. In these embodiments, the adenoviral genome can be a full-length genome or can contain one or more deletions as discussed herein.

Deletion of adenoviral sequences, for example to provide a site for insertion of heterologous sequences or to provide additional capacity for insertion at a different site, can be accomplished by methods well-known to those of skill in the art. For example, for adenoviral sequences cloned in a plasmid, digestion with one or more restriction enzymes (with at least one recognition sequence in the adenoviral insert) followed by ligation will, in some cases, result in deletion of sequences between the restriction enzyme recognition sites. Alternatively, digestion at a single restriction enzyme recognition site within the adenoviral insert, followed by exonuclease treatment, followed by ligation will result in deletion of adenoviral sequences adjacent to the restriction site. A plasmid containing one or more portions of the adenoviral genome with one or more deletions, constructed as described above, can be co-transfected into a bacterial cell along with an adenoviral genome (full-length or deleted) or a plasmid containing either a full-length or a deleted genome to generate, by homologous recombination, a plasmid containing a recombinant genome with a deletion at one or more specific sites. Adenoviral virions containing the deletion can then be obtained by transfection of mammalian cells including but not limited to the stably transformed cells containing the additional fibre gene described herein, with the plasmid containing the recombinant adenoviral genome. The insertion sites may be adjacent to and transcriptionally downstream of endogenous promoters in the adenovirus. An “endogenous” promoter, enhancer, or control region is native to or derived from adenovirus. Restriction enzyme recognition sequences downstream of given promoters that can be used as insertion sites, can be easily determined by one of skill in the art from knowledge of part or all of the sequence of adenoviral genome into which the insertion is desired. Alternatively, various in vitro techniques are available to allow for insertion of a restriction enzyme recognition sequence at a particular site, or for insertion of heterologous sequences at a site that does not contain a restriction enzyme recognition sequence. Such methods include, but are not limited to, oligonucleotide-mediated heteroduplex formation for insertion of one or more restriction enzyme recognition sequences (see, for example, Zoller et al. (1982) Nucleic Acids Res. 10:6487-6500; Brennan et al. (1990) Roux's Arch. Dev. Biol. 199:89-96; and Kunkel et al. (1987) Meth. Enzymology 154:367-382) and PCR-mediated methods for insertion of longer sequences. See, for example, Zheng et al. (1994) Virus Research 31:163-186.

Expression of a heterologous sequence inserted at a site that is not downstream from an endogenous promoter also can be achieved by providing, with the heterologous sequence, a transcriptional regulatory sequences that are active in eukaryotic cells. Such transcriptional regulatory sequences can include cellular promoters such as, for example (DHFR promoter), the viral promoters such as, for example, herpesvirus, adenovirus and papovavirus promoters and DNA copies of retroviral long terminal repeat (LTR) sequences. In such embodiments, the heterologous gene is introduced in an expression construct in which the heterologous gene is operatively linked to such transcriptional regulatory elements.

In specific exemplary embodiments, PCV2 ORF2 gene is placed under the control of a promoter, such as for example, the CMV promoter in order to provide constitutive transcription. In a PAdV3-based viral vector, continued translation of the recombinant PCV2 ORF2 mRNA can be achieved by placing the PCV2 ORF2 gene downstream of the PAdV-3 MLP/TPL sequence. It should be understood that preparation of the recombinant adenoviral vectors includes propagation of the cloned adenoviral genome as a plasmid and rescue of the infectious virus from plasmid-containing cells.

The presence of viral nucleic acids can be detected by techniques known to one of skill in the art including, but not limited to, hybridization assays, polymerase chain reaction, and other types of amplification reactions. Similarly, methods for detection of proteins are well-known to those of skill in the art and include, but are not limited to, various types of immunoassay, ELISA, Western blotting, enzymatic assay, immunohistochemistry, etc. Diagnostic kits comprising the nucleotide sequences of the invention may also contain reagents for cell disruption and nucleic acid purification, as well as buffers and solvents for the formation, selection and detection of hybrids. Diagnostic kits comprising the polypeptides or amino acid sequences of the invention may also comprise reagents for protein isolation and for the formation, isolation, purification and/or detection of immune complexes.

In addition to the PCV2 ORF2, other exogenous (i.e., foreign) nucleotide sequences can be incorporated into the adenovirus. These other exogenous sequences can consist of one or more gene(s) of interest or other nucleotide sequences that are not genes but have other functions of therapeutic interest. In the context of the present invention, a nucleotide sequence or gene of interest can code either for an antisense RNA, short hairpin RNA, a ribozyme or for an mRNA which will then be translated into a protein of interest. Such a nucleotide sequence or gene may comprise genomic DNA, complementary DNA (cDNA) or of mixed type (minigene, in which at least one intron is deleted). The nucleotide sequence or gene can encode a regulatory or therapeutic function, a mature protein, a precursor of a mature protein, in particular a precursor that comprises a signal peptide, a chimeric protein originating from the fusion of sequences of diverse origins, or a mutant of a natural protein displaying improved or modified biological properties. Such a mutant may be obtained by, deletion, substitution and/or addition of one or more nucleotide(s) of the gene coding for the natural protein, or any other type of change in the sequence encoding the natural protein, such as, for example, transposition or inversion.

The gene that is being delivered by the vector may be placed under the control of elements (DNA control sequences) suitable for its expression in a host cell. Suitable DNA control sequences are understood to mean the set of elements needed for transcription of a gene into RNA (antisense RNA or mRNA) and for the translation of an mRNA into protein. For example, these elements would include at least a promoter. The promoter may be a constitutive promoter or a regulatable promoter, and can be isolated from any gene of eukaryotic, prokaryotic or viral origin, and even adenoviral origin. Alternatively, it can be the natural promoter of the gene of interest. Generally speaking, a promoter used in the present invention may be modified so as to contain regulatory sequences. Exemplary promoters may include tissue specific promoters when the gene is to be targeted to a given tissue type. Other conventional promoters that may be used include but are not limited to the HSV-I TK (herpesvirus type 1 thymidine kinase) gene promoter, the adenoviral MLP (major late promoter), the RSV (Rous Sarcoma Virus) LTR (long terminal repeat), the CMV immediate early promoter, SV-40 immediate early promoter, and the PGK (phosphoglycerate kinase) gene promoter, for example, permitting expression in a large number of cell types.

The viral vectors or indeed a pharmaceutical composition comprising the viral vectors can additionally include at least one immunogen from at least one additional pig pathogen, e.g.: Porcine Reproductive and Respiratory Syndrome (PRRS), Mycoplasma hyopneumoniae, Actinobacillus pleuropneumoniae, E. coli, Bordetella bronchiseptica, Pasteurella multocida, Erysipelothrix rhusiopathiae, Pseudorabies, Hog cholera, Swine Influenza, and Porcine Parvovirus (PPV). Thus, vector-based compositions can include at least one immunogen from at least one additional pig pathogen, such as a vector expressing a sequence from this pathogen, wherein the vector is also capable of expressing the PCV-2 ORF2 described above. Alternatively, the vaccine composition can be made of one vector component that expresses the PCV2 ORF2 as described herein and a second component that can either be a recombinant vector expressing a second immunogen or the second component is a composition that contains the isolated immunogen that has been isolated from another source

While much of the present description relates to porcine adenoviruses as exemplary vaccine vectors, the vector can comprise any viral vector including, e.g., a virus such as a herpesvirus including pig herpes viruses, including Aujeszky's disease virus (also known as pseudorabies virus), an adenovirus including a porcine adenovirus or a human adenovirus of any serotype, a poxvirus, including a vaccinia virus, an avipox virus, a canarypox virus, a racoonpox and a swinepox virus, and the like.

In certain preferred embodiments the vaccines of the present invention are prepared to vaccinate swine against diseases other than and in addition to PMWS in those animals. For example, the vaccines may be directed to pseudorabies virus (PRV) gp50; transmissible gastroenteritis virus (TGEV) S gene; porcine rotavirus VP7 and VP8 genes; genes of porcine respiratory and reproductive syndrome virus (PRRS); genes of porcine epidemic diarrhea virus; genes of hog cholera virus; genes of porcine parvovirus; and genes of foot-and-mouth disease virus; genes of porcine influenza virus; and other genes associated with porcine circovirus in addition to PCV2 ORF2.

It should be understood that while in some circumstances it might be desirable to incorporate the whole gene into the vector, other vectors can be constructed that comprise only a portion of the nucleotide sequences of genes can be used (where these are sufficient to generate a protective immune response or a specific biological effect) rather than the complete sequence as found in the wild-type organism. Where the genes contain a large number of introns, a cDNA may be preferred.

As noted above, the gene may be inserted under the control of a suitable promoter. In addition the vector also may comprise enhancer elements and polyadenylation sequences. Promoters and polyadenylation sequences which provide successful expression of foreign genes in mammalian cells and construction of expression cassettes, are known in the art, for example in U.S. Pat. No. 5,151,267, the disclosures of which are incorporated herein by reference.

The term “expression cassette” refers to a natural or recombinantly produced nucleic acid molecule that is capable of expressing a gene or genetic sequence in a cell. An expression cassette typically includes a promoter (allowing transcription initiation), and a sequence encoding one or more proteins or RNAs. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences. Optionally, the expression cassette may include a gene or partial gene sequence that is not translated into a protein. The nucleic acid can effect a change in the DNA or RNA sequence of the target cell. This can be achieved by hybridization, multi-strand nucleic acid formation, homologous recombination, gene conversion, RNA interference or other yet to be described mechanisms

The viral vectors may comprise more than one foreign gene. The methods of the invention are preferably used to provide protection against PCV2 associated disease in pigs. While exemplary embodiments of the present invention are such that the heterologous nucleotide (also referred to herein in as heterologous nucleic acid) is one which encodes a protein, it should be understood that the heterologous nucleotide may in fact be any polynucleotide containing a sequence whose presence or transcription in a cell is desired. Thus the vectors may be used to to deliver any polynucleotide that, for example, causes sequence-specific degradation or inhibition of the function, transcription, or translation of a gene.

The immunogen compositions other than the modified PCV2 ORF2 can be recombinantly produced or extracted from natural sources or may be chemically synthesized. For example, the immunogen compositions other than the modified PCV2 ORF2, can be isolated and/or purified from infected or transfected cells; for instance, to prepare compositions for administration to pigs; however, in certain instances, it may be advantageous not to isolate and/or purify an expression product from a cell; for instance, when the cell or portions thereof enhance the immunogenic effect of the polypeptide. Protein purification and/or isolation teahcniques used to achieve this are well known to those of skill in the art and in general, can include: precipitation by taking advantage of the solubility of the protein of interest at varying salt concentrations, precipitation with organic solvents, polymers and other materials, affinity precipitation and selective denaturation; column chromatography, including high performance liquid chromatography (HPLC), ion-exchange, affinity, immunoaffinity or dye-ligand chromatography; immunoprecipitation, gel filtration, electrophoretic methods, ultrafiltration and isoelectric focusing, and their combinations.

It has previously been shown that a modified rPAdV-gp55 grown in PK-15 cells when administered to commercially available Large White Pigs by sub-cutaneous or oral routes completely protected pigs from lethal challenge with CSFV when given as subcutaneous injection or by the oral route. In the context of the present invention a similar approach may be taken to administer a modified rPAdV-PCV2 ORF2 either alone or in combination with gp55 or some other antigen to confer an effective immunity or vaccination of the pigs against disease.

In order to allow the PCV2 ORF2 to be taken up by as many tissues in the animal as possible, or to specifically target a given tissue, the PAdV may be modified to contain a fibre gene from more than one serotype of PAdV (e.g., the recombinant vaccine that contains the PCV2 ORF2 also contains the gene for PAdV3 fibre and PAdV4 fibre). In this manner, a modified PCV2 ORF-2 containing vaccine that contains both the PAdV-3 and PAdV-4 fibre proteins will target to a wider variety of tissues in the pig than the unmodified vaccine, and as a consequence generate a more extensive immune response in the host.

Specifically contemplated herein are pharmaceutical compositions comprising a therapeutically effective amount of a recombinant adenovirus vector, recombinant adenovirus or recombinant protein, prepared according to the methods of the invention, in combination with a pharmaceutically acceptable vehicle and/or an adjuvant. Such a pharmaceutical composition can be prepared and dosages determined according to techniques that are well-known in the art. The pharmaceutical compositions of the invention can be administered by any known administration route including, but not limited to, systemically (for example, intravenously, intratracheally, intravascularly, intrapulmonarilly, intraperitoneally, intranasally, parenterally, enterically, intramuscularly, subcutaneously, intratumorally or intracranially), by oral administration, by aerosolization or intrapulmonary instillation. Administration can take place in a single dose or in doses repeated one or more times after certain time intervals. The appropriate administration route and dosage will vary in accordance with the situation (for example, the individual being treated, the disorder to be treated or the gene or polypeptide of interest), but can be determined by one of skill in the art.

In specific embodiments, female pigs will be inoculated with a viral vector composition that comprises a nucleic acid that expresses at least one therapeutic protein, i.e., a modified PCV2 ORF2 that when expressed does not localize to the nucleus of an infected cell but rather it lacks the nuclear localization signal and hence is released into the cytoplasm of the cell. The animal may be inoculated prior to breeding; and/or prior to serving, and/or during gestation (or pregnancy); and/or prior to the perinatal period or farrowing; and/or repeatedly over a lifetime, to prevent myocarditis and/or abortion and/or intrauterine infection associated with PCV-2, as well as post-weaning multisystemic wasting syndrome and other pathologic sequelae associated with PCV-2; or, to elicit an immunogenic or protective response against PCV-2 and thereby prevent any disease associated with PCV-2 infection. Such diseases include but are not limited to post-weaning multisystemic wasting syndrome and/or porcine dermatitis and nephropathy syndrome and/or myocarditis and/or abortion and/or intrauterine infection associated with porcine circovirus-2 and/or other pathologic sequelae associated with PCV-2. While the present invention is exemplified by treatment of what is currently termed “post-weaning multisystemic wasting syndrome” it should be understood that the compositions and methods of the present invention will be useful in the treatment of any disease associated with PCV-2 infection and a beneficial result will be the amelioration of any of the symptoms associated with that disease including secondary infections caused by bacterial infections, such as Glasser disease (Haemophilus parasuis), Pulmonary Pasteurellosis, Colibacilosis and Salmonellosis and the like. Other symptoms include wasting, dyspnea, and paleness, combined with pathological findings of enlarged lymph nodes, interstitial pneumonia, and nephritis. Lymphocyte depletion and histiocytic to granulomatous inflammation in lymphoid tissues and certain organs are the main histological changes seen in PCV-2 associated diseases. The methods and compositions of the present invention are used to prevent, inhibit, or otherwise reduce or decrease the effects of these symptoms.

In another embodiment, piglets are inoculated within the first weeks of life, e.g., inoculation at one and/or two and/or three and/or four and/or five weeks of life. More preferably, piglets are first inoculated within the first week of life or within the third week of life (e.g., at the time of weaning). Even more advantageous, such piglets are then boosted two (2) to four (4) weeks later (after being first inoculated). The piglets may be from vaccinated or unvaccinated females. Thus, both offspring, as well as female pig can be administered the compositions of the invention in order to increase the life expectancy of the piglets and their mothers.

The invention further provides for methods of treatment in which a therapeutically effective amount of a recombinant adenoviral vector (e.g., a PAdV-3 adenoviral vector) that contains PCV2 ORF2 as the therapeutic antigen.

The antigens other than the modified PCV2 ORF2 that are used in combination with the modified PCV2 ORF2 can be either native or recombinant antigenic polypeptides or fragments.

They can be partial sequences, full-length sequences, or even fusions (e.g., having appropriate leader sequences for the recombinant host, or with an additional antigen sequence for another pathogen). The preferred antigenic polypeptide to be expressed by the virus systems of the present invention contain full-length (or near full-length) sequences encoding antigens. Alternatively, shorter sequences that are antigenic (i.e., encode one or more epitopes) can be used. The shorter sequence can encode a “neutralizing epitope,” which is defined as an epitope capable of eliciting antibodies that neutralize virus infectivity in an in vitro assay. Preferably the peptide should encode a “protective epitope” that is capable of raising in the host a “protective immune response;” i.e., an antibody- and/or a cell-mediated immune response that protects an immunized host from infection.

In addition, any of the vaccines in the present invention also may comprise an adjuvant. An “adjuvant” is any substance added to a vaccine to increase the immunogenicity of the vaccine. The use of adjuvants in vaccine compositions are well known in the art: for example, bovine serum albumin (BSA), human serum albumin (HSA) and keyhole limpet hemocyanin (KLH). Some adjuvants are believed to enhance the immune response by slowly releasing the antigen, while other adjuvants are strongly immunogenic in their own right and are believed to function synergistically. Known vaccine adjuvants include, but are not limited to, oil and water emulsions (for example, complete Freund's adjuvant and incomplete Freund's adjuvant), Corynebacterium parvum, Bacillus Calmette Guerin, aluminum hydroxide, glucan, dextran sulfate, iron oxide, sodium alginate, Bacto-Adjuvant, certain synthetic polymers such as poly amino acids and co-polymers of amino acids, saponin, “REGRESSIN” (Vetrepharm, Athens, Ga.), “AVRIDINE” (N,N-dioctadecyl-N′,N′-bis(2-hydroxyethyl)-propanediamine), paraffin oil, muramyl dipeptide and the like.

Genes for desired antigens or coding sequences thereof which can be inserted include those of organisms which cause disease in mammals, particularly bovine pathogens such as foot-and-mouth disease virus, bovine rotavirus, bovine coronavirus, bovine herpes virus type 1, bovine respiratory syncytial virus, bovine parainfluenza virus type 3 (BPI-3), bovine diarrhea virus, Pasteurella haemolytica, Haemophilus somnus and the like. Genes encoding antigens of human pathogens also may be useful in the practice of the invention. The vaccines of the invention carrying foreign genes or fragments can also be orally administered in a suitable oral carrier, such as in an enteric-coated dosage form. Oral formulations include such normally-employed excipients as, for example, pharmaceutical grades of mannitol, lactose, starch, magnesium stearate, sodium saccharin cellulose, magnesium carbonate, and the like. Oral vaccine compositions may be taken in the form of solutions, suspensions, tablets, pills, capsules, sustained release formulations, or powders, containing from about 10% to about 95% of the active ingredient, preferably about 25% to about 70%. Oral and/or intranasal vaccination may be preferable to raise mucosal immunity (which plays an important role in protection against pathogens infecting the respiratory and gastrointestinal tracts) in combination with systemic immunity.

In addition, the vaccine can be formulated into a suppository. For suppositories, the vaccine composition will include traditional binders and carriers, such as polyalkaline glycols or triglycerides. Such suppositories may be formed from mixtures containing the active ingredient in the range of about 0.5% to about 10% (w/w), preferably about 1% to about 2%.

Protocols for administering to animals the vaccine composition(s) of the present invention are within the skill of the art in view of the present disclosure. Those skilled in the art will select a concentration of the vaccine composition in a dose effective to elicit an antibody and/or T-cell mediated immune response to the antigenic fragment or another type of therapeutic or prophylactic effect. Within wide limits, the dosage is not believed to be critical. The timing of administration may also be important. For example, a primary inoculation preferably may be followed by subsequent booster inoculations if needed. It may also be preferred, although optional, to administer a second, booster immunization to the animal several weeks to several months after the initial immunization. To insure sustained high levels of protection against disease, it may be helpful to readminister a booster immunization to the animals at regular intervals, for example once every several years. Alternatively, an initial dose may be administered orally followed by later inoculations, or vice versa. Preferred vaccination protocols can be established through routine vaccination protocol experiments.

The dosage for all routes of administration of in vivo recombinant virus vaccine depends on various factors including, the size of host/patient, nature of infection against which protection is needed, carrier and the like and can readily be determined by those of skill in the art. By way of non-limiting example, a dosage of between 10² pfu and 10¹⁵ pfu, preferably between 10⁴ and 10¹³ pfu, more preferably between 10⁵ to 10¹¹ pfu and the like can be used. As with in vitro subunit vaccines, additional dosages can be given as determined by the clinical factors involved.

The invention also includes a method for providing gene delivery to a mammal, and particularly to pigs, to control a gene deficiency, to provide a therapeutic gene or nucleotide sequence and/or to induce or correct a gene mutation. The method can be used, for example, in the treatment of conditions including, but not limited to hereditary disease, infectious disease, cardiovascular disease, and viral infection. These kinds of techniques are currently being used by those of skill in the art for the treatment of a variety of disease conditions. Examples of foreign genes, nucleotide sequences or portions thereof that can be incorporated for use in a conventional gene therapy include, cystic fibrosis transmembrane conductance regulator gene, human minidystrophin gene, alpha-1-antitrypsin gene, genes involved in cardiovascular disease, and the like.

For the purposes of the present invention, the vectors, cells and viral particles prepared by the methods of the invention may be introduced into a subject either ex vivo, (i.e., in a cell or cells removed from the patient) or directly in vivo into the body to be treated.

### EXAMPLES

### Example 1

FIG. 1 shows an exemplary protocol for the production to the recombinant viral vectors used herein. A truncated PCV2 ORF2 gene was PCR amplified from a full length PCV2 ORF2 gene cloned in a plasmid as template using 5′ and 3′ gene specific primers. The 5′ PCR primer was specifically designed to bind 127 by downstream of the start of the PCV2 ORF2 gene (which allows for the deletion of the NLS) and also introduced a signal sequence which incorporated in-frame onto the 5′ end of the final PCR product. To facilitate cloning of the product, both 5′ and 3′ primers also introduced the restriction sites BglII and HindIII respectively to the final PCR product.

The PCR amplified product comprising of truncated PCV2 ORF2 gene with signal sequence was then cloned into the BglII and HindIII sites of the expression cassette within the PAV3 RHE plasmid. The recombinant PAV3 RHE plasmid and PAV3 LHE plasmid are then linearized using restriction enzyme which cut specifically within the plasmid backbone sequence (Enzyme ‘X’ and ‘Y’) but not within PAV3 genomic sequence or the inserted DNA.

The linearized PAV3 LHE and PAV3 RHE plasmid DNA which both carry portions of the PAV3 viral genome were co-transfected into porcine cells. Both DNA fragments have an ˜1 kb region of homologous overlapping PAV3 sequence which directs homologous recombination to occur and reconstitute a competent full length recombinant PAV3 viral genome with the inserted DNA.

Successive passage of transfected cells results in the enrichment of infective particles which appear as viral plaques. These represent recombinant PAV3 viruses expressing a truncated PCV2 ORF2 protein with a 5′ in frame signal sequence.

While the above example demonstrates insertion into the PAV-3 RHE, it should be understood that the insertions can be made in other non-essential regions of the PAV3 genome.

### Example 2

In order to test the efficacy of the vaccines of the present invention, groups of piglets were given two doses of either a vaccine based on the modified PCV2-ORF2 as described herein or a vaccine that contains unmodified PCV2-ORF2 and the susceptibility of the pigs to a challenge with PCV2 determined. In addition, the ability of the modified vaccine to induce neutralising antibody and to give protection when administered by the oral route will be tested.

The present example describes a study designed to evaluate protection afforded weaned piglets by two doses of three different recombinant porcine adenovirus serotype 3 vaccine candidates containing open reading frame 2 from porcine circovirus 2 (PCV2) derived from a synthetic consensus sequence. The parent recombinant is designated rPAV-3 PCV2 mORF2. Protection will be evaluated following challenge of vaccinated piglets with American Type Culture Collection (ATCC)PCV2 isolate TBA and measuring the effect on viremia as measured by virus isolation, body weights, post challenge rectal temperatures, lymph node histopathology and virus isolation from lymphoid tissue, kidney, thymus, lungs and peyers patches at necropsy.

A herd of 60 piglets of 21 days of age from a PCV2-free herd are used in the study. The following table sets forth an exemplary vaccination protocol for the herd.

STUDY DESIGN

| Trt. | Treatment | Number | Vaccination | | | Challenge |
|---|---|---|---|---|---|---|
| No. | Group | of pigs | Days | Dose | Route | Necropsy |
| T1 | PBS | 10-15 | 0.14 | 2.0 ml | IM | Challenge Day 28 |
| | | | | | | Necropsy Day 49 |
| T2 | rPAV-3 PCV2 | 10-15 | 0.14 | 1 × 10⁸/ | IM | Challenge Day 28 |
| | mORF2 V1 | | | 2.0 ml | | Necropsy Day 49 |
| T3 | rPAV-3 PCV2 | 10-15 | 0.14 | 1 × 10⁸/ | IM | Challenge Day 28 |
| | mORF2 V2 | | | 2.0 ml | | Necropsy Day 49 |
| T4 | fPAV-3 PCV2 | 10-15 | 0.14 | 1 × 10⁸/ | IM | Challenge Day 28 |
| | mORF2 V3 | | | 2.0 ml | | Necropsy Day 49 |

More specifically, recently weaned 21 (+/−4 days) day old piglets will be sourced from a PCV1 and PCV2a and PCV2b negative swine herd and transported to the trial site. Piglets will be individually identified by ear tags. Animal waste will be captured in tanks and disinfected prior to release in a lagoon. Clinical observations on piglets will be recorded once daily through the end of the study. Piglets will be evaluated for depression, lethargy, increased respiratory rate, respiratory distress, being moribund, and death.

On Day 0, blood samples (2.0 to 4.0 ml per piglet), body weights and rectal temperatures will be collected from each piglet. Piglets in treatment group T1 will receive placebo, piglets in T2 will be vaccinated by the intramuscular route with the rPAV-3 PCV2 mORF2 V1, piglets in T3 will be vaccinated by the IM route with the rPAV-3 PCV2 mORF2 V2 and piglets in T4 will be vaccinated by the IM route with the rPAV-3 PCV2 mORF2 V3.

On Day 14, blood samples (2.0 to 4.0 ml per piglet), body weights, and rectal temperatures will be collected from each piglet in treatment groups T1, T2, T3 and T4. Also on Day 14, piglets in treatment groups T1 will receive placebo, piglets in T2 will be vaccinated by the intramuscular route with the rPAV-3 PCV2 mORF2 V1, piglets in T3 will be vaccinated by the IM route with the rPAV-3 PCV2 mORF2 V2 and piglets in T4 will be vaccinated by the IM route with the rPAV-3 PCV2 mORF2 V3.

On Day 28, blood samples (2.0 to 4.0 ml per piglet), body weights, and rectal temperatures will be collected from piglets in treatment groups T1, T2, T3 and T4. Also on Day 28, piglets in treatment groups T1, T2, T3 and T4 will be exposed by the intranasal route to 1.0 ml of challenge inoculum of the agreed PCV2 virus isolate at the agreed target dose. The challenge inoculum will be titered prior to challenge and documented in a note to file.

On Day 35, blood samples (2.0 to 4.0 ml per piglet), body weights, and rectal temperatures will be collected from piglets in treatment groups T1, T2, T3 and T4. On Day 42, blood samples (2.0 to 4.0 ml per piglet), body weights, and rectal temperatures will be collected from piglets in treatment groups T1, T2, T3 and T4. On Day 49, blood samples (2.0 to 4.0 ml per piglet), body weights, and rectal temperatures will be collected from piglets in treatment groups T1, T2, T3 and T4.

Also on Day 49, all piglets in treatment groups T1, T2 and T3 will be euthanized, necropsied, and lymph node samples will be stored in formalin for possible later histopathological examination. Lung, kidney, thymus, lymphoid and peyers patch tissue samples will be obtained for PCV2 virus isolation.

PCV2 virus isolation testing is performed on serum samples collected from piglets on Days 28, 35, 42, and 49 will be analyzed for PCV2 virus by virus isolation. Antibody levels in the serum is tested on serum samples collected from piglets on Days 0, 14, 28, 35, 42 and 49 and analysed by ELISA for antibody titers against PCV2 virus. Serum samples collected from piglets on Days 0, 14, 28, 35, 42 and 49 will be stored for possible later analysis for ELISA titers against and PAV3 virus.

Virus isolations will be performed with serum samples collected on Days 28 35, 42 and 49. The serum samples collected from piglets on days 0, 14, 21, 28 35, 42 and 49 will be tested for the presence of antibodies to PCV2 by using commercially available IgG PCV2-ELISA kits to (Ingezim PCV IgG® (Ingenasa, Madrid, Spain). The various serum samples also will be stored for possible future testing for the presence of PCV2 genome by PCR assay.

The sizes of the lymph nodes (superficial, inguinal, mediastinal, tracheobronchial, and mesenteric) ranging from 0 (normal) to 3 (four times the normal size) will be estimated and recorded.

It is expected that the vaccine containing the modified PCV2 ORF2 will produce a greater immunity than that seen when the unmodified PCV2 ORF-2 based vaccine is administered. It is predicted that the vaccine containing the modified PCV2 ORF2 will completely protect pigs at a dosage that is less than a dosage of the unmodified PCV2 ORF2. Such beneficial effects are monitored after subcutaneous injection or by the oral route.

### Example 3

### Trial Data

In order to evaluate protection afforded weaned piglets by the modified PCV2 ORF2 based vaccines a trial was conduct. In this trial, two doses of three different recombinant porcine adenovirus serotype 3 vaccine candidates containing PCV2 ORF2 derived from a synthetic consensus sequence were used. The parent recombinant was designated rPAV-3 PCV2 mORF2. Protection was evaluated following challenge of vaccinated piglets with PCV2 and measuring the effect on viremia as measured by virus isolation and clinical signs.

The three candidate vaccines were:

(1) PAdV3-PCV2ORF2 full-length in which the PCV2 ORF 3 was unmodified;

(2) PAdV3-PCV2ORF2 Truncated in which the PCV2 ORF2 nuclear localization signal has been removed and

(3) PMV3-OCV2ORF2 Secreted in which the PCV2 ORF2 has had the NLS removed and replaced by a hydrophobic signal sequence and cleavage site.

In the protocol, 3 week old piglets were vaccinated with either (1) PAdV3-PCV2ORF2 full length; (2)PAdV3-PCV2ORF2 truncated, (3) PAdV3-PCV2ORF2 secreted or with phosphate buffered saline (control). At 5 weeks of age all pigs received a second (boost) vaccination. All of the vaccinations were intramuscular (IM). At 7 weeks of age all pigs were challenged with PCV2 and the trial was terminated at 10 weeks of age.

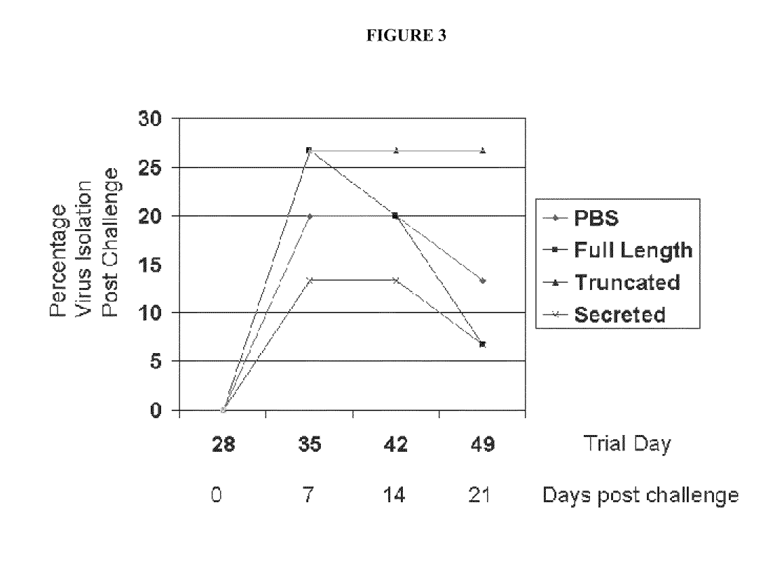

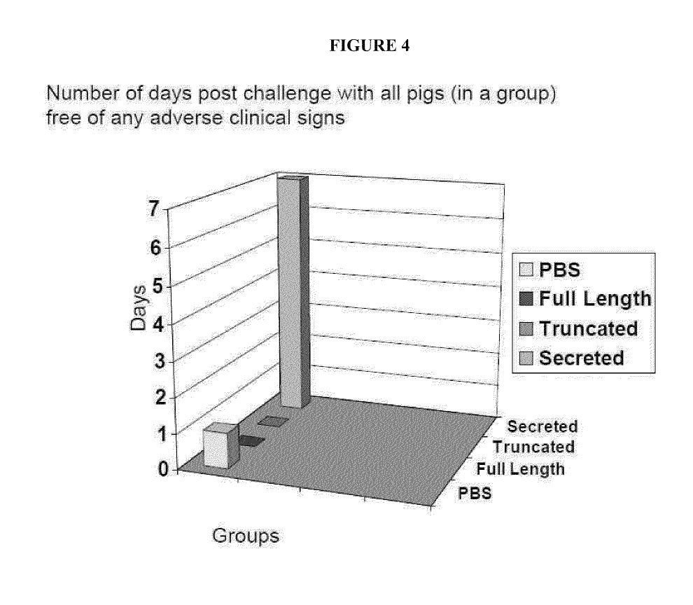

The data from this trial are shown in FIG. 3 (showing virus isolation) and FIG. 4 (showing presence of clinical symptoms). As can be seen from the data in these figures, both virus isolation (post challenge) and clinical signs, the secreted version (no. 3 above) was most effective as a vaccine against PCV2. Indeed, in each of the group of piglets treated with PBS, full length PCV2ORF and truncated PCVS ORF, the pigs developed clinical symptoms of PCV2 within the first day whereas the pigs that had been vaccinated with PAdV3-OCV2ORF2 Secreted in which the PCV2 ORF2 has had the NLS removed and replaced by a hydrophobic signal sequence and cleavage site had not developed any adverse clinical symptoms at day 7 post-challenge.

What is claimed is:

1. A recombinant expression vector comprising a nucleic acid sequence that encodes a modified PCV2 ORF2 operably linked to a promoter, wherein
a. the modified PCV2 ORF2 is one in which the nuclear localization signal of wild-type PCV2 ORF2 has been removed or modified to allow secretion of truncated ORF2 protein upon expression; or
b. the modified PCV2 ORF2 is one in which the nuclear localization signal has been removed and replaced with a signal hydrophobic signal that directs expression of the PCV2 ORF2 on the cell surface of an infected cell.

2. The recombinant expression vector of claim 1, wherein the nuclear localization signal of said ORF2 has been replaced with a hydrophobic signal sequence and cleavage site.

3. The recombinant expression vector of claim 1, wherein the nuclear localization signal of said ORF2 is replaced with the signal sequence selected from the group consisting of chicken gamma interferon, porcine gamma interferon, and the HA protein of influenza virus.

4. The recombinant expression vector of claim 1, wherein said vector is a viral vector.

5. The recombinant expression vector of claim 4, wherein said viral vector is selected from the group consisting of an adenoviral vector, an adenoassociated viral vector, a lentiviral vector, a herpes viral vector, a pox viral vector.

6. The recombinant expression vector of claim 5, wherein said adenoviral vector is a porcine adenoviral vector selected from the group consisting of PAdV1, PAdV2, PAdV3, PAdV4, and PAdV5.

7. The recombinant expression vector of claim 6, wherein said porcine adenoviral vector is PAdV3.

8. The vaccine of claim 7, wherein said PAVd3 is a replication competent PAdV3

9. The recombinant expression vector of claim 7, wherein said nucleic acid sequence that encodes said modified PCV ORF2 is inserted into a non-essential sequence in PAdV3.

10. The recombinant expression vector of claim 9, wherein said non-essential sequence of PAdV-3 is selected from the group consisting of the E3 region, ORF 1-2 and 4-7 of E4, the region between the end of E4 and the ITR of the porcine adenovirus genome.

11. The recombinant expression vector of claim 7, wherein said PAdV3 is a recombinant PAdV3 comprising a fibre gene native to said PAdV3 and further comprising a second fibre gene that is heterologous to said adenovirus, wherein said second fibre gene is acquired by said recombinant adenovirus by growth of said recombinant adenovirus in a cell line that stably expresses said second fibre gene.

12. The recombinant expression vector of claim 1, wherein said nucleic acid comprises the sequence of SEQ ID NO:1, SEQ ID NO:3 or SEQ ID NO:5.

13. The recombinant expression vector of claim 1, further comprising a nucleic acid that encodes another antigen for eliciting an immune response in pigs.

14. A composition comprising a first recombinant expression vector of claim 1, and a second recombinant expression vector that comprises an additional antigen for eliciting an immune response in pigs.

15. A vaccine for eliciting a protective response against porcine circovirus (PCV2) infection in pigs comprising a veterinarily acceptable vehicle or excipient and a recombinant expression vector of claim 1.

16. A vaccine for eliciting a protective response against PCV2 infection in pigs comprising a composition of claim 14.

17. The vaccine of claim 15, further comprising one or more additional antigen for vaccination of pigs wherein said additional one or more antigen is provided as a protein component in the veterinarily acceptable vehicle or excipient of said vaccine.

18. A vaccine for the protection of pigs against diseases caused by PCV-2 ORF2, said vaccine comprising a recombinant virus vector comprising a promoter operably linked to a hydrophobic signal sequence comprising a nucleic acid that encodes a membrane anchoring domain, a multiple cloning site for insertion of a modified PCV-2 ORF2 in frame with said hydrophobic signal sequence, a polyadenylation signal; and a viral genome, wherein said modified PCV-2 ORF2 lacks a nuclear localization signal.

19. The vaccine of claim 18, wherein said vector further comprises a cleavage sequence immediately upstream of the cloning site for modified PCV-2 ORF2, wherein the PCV-2 ORF 2 expression product from said vector produces a soluble gene product.

20. A vaccine for the protection of pigs from PCV-2 associated disorder, said vaccine comprising a recombinant porcine adenovirus 3 vector comprising a promoter operably linked to a hydrophobic signal sequence comprising a nucleic acid that encodes a membrane anchoring domain, and a nucleic acid that encodes a truncated PCV2 ORF2 that lacks a NLS sequence inserted in frame with said hydrophobic signal sequence, a polyadenylation signal; and a porcine adenovirus 3 genome.

21. The vaccine of claim 17, wherein said vaccine is formulated for aerosol administration.

22. The vaccine of claim 17, wherein said vaccine is formulated for oral, nasal, intramuscular, subcutaneous, or intradermal delivery.

23. A method for eliciting an immune response in a porcine subject comprising administering a vaccine of claim 17 to the porcine subject in an amount effective to elicit a protective immune response in said porcine subject.

24. A method for reducing viral load of porcine circovirus 2 (PCV2) in a pig comprising inducing an immunological or immunogenic response against PCV2 in the pig comprising administering to the pig a composition comprising a pharmaceutically or veterinarily or medically acceptable carrier and an expression vector of claim 1.

25. The method of claim 24, wherein the administering is prior to breeding.

26. The method of claim 24, wherein the pig is a pregnant female pig.


SEQUENCE LISTING (106 sequences)

SEQ ID NO: 1 - DNA, 657 bp, Artificial Sequence
  Chicken gamma IFN-PCV ORF2
atgacttgccagacttacaacttgtttgttctgtctgtcatcatgatttattatggacatactgcaagtagtctaaatcttggcatcttcaacacccgcctctcccgcaccttcggatatactgtcaaggctaccacagtcacaacgccctcctgggcggtggacatgatgagatttaatattgatgactttgttcccccgggaggggggaccaacaaaatctctataccctttgaatactacagaataagaaaggttaaggttgaattctggccctgctccccaatcacccagggtgacaggggagttggatccagtgctgttattctagatgataactttgtaacaaaggccacagccctaacctacgacccctatgtaaactactcctcccgccataccataccccagcccttctcctaccactcccgctatttcacccccaaaccggtccttgatagcacaatcgattacttccaacccaataacaaaagaaatcaactctggctaagactacaaacctctgcaaatgtggaccacgtaggcctcggcactgcgttcgaaaacagtaaatacgaccaggactacaatatccgtgtaaccatgtatgtacaattcagagaatttaatcttaaagaccccccacttaaaccctaa

SEQ ID NO: 2 - protein, 218 aa, Artificial Sequence
  Chicken gamma IFN-PCV ORF2
MTCQTYNLFVLSVIMIYYGHTASSLNLGIFNTRLSRTFGYTVKATTVTTPSWAVDMMRFNIDDFVPPGGGTNKISIPFEYYRIRKVKVEFWPCSPITQGDRGVGSSAVILDDNFVTKATALTYDPYVNYSSRHTIPQPFSYHSRYFTPKPVLDSTIDYFQPNNKRNQLWLRLQTSANVDHVGLGTAFENSKYDQDYNIRVTMYVQFREFNLKDPPLKP

SEQ ID NO: 3 - DNA, 645 bp, Artificial Sequence
  Porcine gamma IFN-PCV ORF2
atgagttatacaacttatttcttagcttttcagctttgcgtgactttgtgtttttctggctcttactgcggcatcttcaacacccgcctctcccgcaccttcggatatactgtcaaggctaccacagtcacaacgccctcctgggcggtggacatgatgagatttaatattgatgactttgttcccccgggaggggggaccaacaaaatctctataccctttgaatactacagaataagaaaggttaaggttgaattctggccctgctccccaatcacccagggtgacaggggagttggatccagtgctgttattctagatgataactttgtaacaaaggccacagccctaacctacgacccctatgtaaactactcctcccgccataccataccccagcccttctcctaccactcccgctatttcacccccaaaccggtccttgatagcacaatcgattacttccaacccaataacaaaagaaatcaactctggctaagactacaaacctctgcaaatgtggaccacgtaggcctcggcactgcgttcgaaaacagtaaatacgaccaggactacaatatccgtgtaaccatgtatgtacaattcagagaatttaatcttaaagaccccccacttaaaccctaa

SEQ ID NO: 4 - protein, 214 aa, Artificial Sequence
  Porcine gamma IFN-PCV ORF2
MSYTTYFLAFQLCVTLCFSGSYCGIFNTRLSRTFGYTVKATTVTTPSWAVDMMRFNIDDFVPPGGGTNKISIPFEYYRIRKVKVEFWPCSPITQGDRGVGSSAVILDDNFVTKATALTYDPYVNYSSRHTIPQPFSYHSRYFTPKPVLDSTIDYFQPNNKRNQLWLRLQTSANVDHVGLGTAFENSKYDQDYNIRVTMYVQFREFNLKDPPLKP

SEQ ID NO: 5 - DNA, 636 bp, Artificial Sequence
  Human H1N2 HA-PCV ORF2
atgaaagtaaaactactgatcctgttatgtacatttacagctacatatgcagacacaataggcatcttcaacacccgcctctcccgcaccttcggatatactgtcaaggctaccacagtcacaacgccctcctgggcggtggacatgatgagatttaatattgatgactttgttcccccgggaggggggaccaacaaaatctctataccctttgaatactacagaataagaaaggttaaggttgaattctggccctgctccccaatcacccagggtgacaggggagttggatccagtgctgttattctagatgataactttgtaacaaaggccacagccctaacctacgacccctatgtaaactactcctcccgccataccataccccagcccttctcctaccactcccgctatttcacccccaaaccggtccttgatagcacaatcgattacttccaacccaataacaaaagaaatcaactctggctaagactacaaacctctgcaaatgtggaccacgtaggcctcggcactgcgttcgaaaacagtaaatacgaccaggactacaatatccgtgtaaccatgtatgtacaattcagagaatttaatcttaaagaccccccacttaaaccctaa

SEQ ID NO: 6 - protein, 211 aa, Artificial Sequence
  Human H1N2 HA-PCV ORF2
MKVKLLILLCTFTATYADTIGIFNTRLSRTFGYTVKATTVTTPSWAVDMMRFNIDDFVPPGGGTNKISIPFEYYRIRKVKVEFWPCSPITQGDRGVGSSAVILDDNFVTKATALTYDPYVNYSSRHTIPQPFSYHSRYFTPKPVLDSTIDYFQPNNKRNQLWLRLQTSANVDHVGLGTAFENSKYDQDYNIRVTMYVQFREFNLKDPPLKP

SEQ ID NO: 7 - DNA, 702 bp, Porcine circovirus
  misc_feature: Full Length PCV2 ORF2
atgacgtatccaaggaggcgttaccgcagacgaagacaccgcccccgcagccatcttggccagatcctccgccgccgcccctggctcgtccacccccgccaccgttaccgctggagaaggaaaaatggcatcttcaacacccgcctctcccgcaccttcggatatactgtcaaggctaccacagtcacaacgccctcctgggcggtggacatgctgagatttaatattaatgactttgttcccccgggaggggggaccaacaaaatctctataccctttgaatactacagaataagaaaggttaaggttgaattctggccctgctccccaatcacccagggtgacaggggagttggatccagtgctgttattctagatgataactttgtaacaaagaccacagccctaacctacgacccctatgtaaactactcctcccgccataccataacccagcccttctcctaccactcccgctatttcacccccaaaccggtccttgatgggacaatcgattacttccaacccaataacaaaagaaatcaactctggctaagactacaaacctctgcaaatgtggaccacgtaggcctcggcactgcgttcgaaaacagtaaatacgaccaggactacaatatccgtgtaaccatgtatgtacaattcagagaatttaatcttaaagaccccccacttaaaccctaa

SEQ ID NO: 8 - protein, 233 aa, Porcine circovirus
  MISC_FEATURE: Full Length PCV2 ORF2
MTYPRRRYRRRRHRPRSHLGQILRRRPWLVHPRHRYRWRRKNGIFNTRLSRTFGYTVKATTVTTPSWAVDMLRFNINDFVPPGGGTNKISIPFEYYRIRKVKVEFWPCSPITQGDRGVGSSAVILDDNFVTKTTALTYDPYVNYSSRHTITQPFSYHSRYFTPKPVLDGTIDYFQPNNKRNQLWLRLQTSANVDHVGLGTAFENSKYDQDYNIRVTMYVQFREFNLKDPPLKP

SEQ ID NO: 9 - DNA, 126 bp, Porcine circovirus
  misc_feature: Signal sequence of PCV2 ORF2
atgacgtatccaaggaggcgttaccgcagacgaagacaccgcccccgcagccatcttggccagatcctccgccgccgcccctggctcgtccacccccgccaccgttaccgctggagaaggaaaaat

SEQ ID NO: 10 - protein, 42 aa, Porcine Circovirus
  MISC_FEATURE: Signal sequence of PCV2 ORF2
MTYPRRRYRRRRHRPRSHLGQILRRRPWLVHPRHRYRWRRKN

SEQ ID NO: 11 - DNA, 81 bp, Gallus gallus
atgacttgccagacttacaacttgtttgttctgtctgtcatcatgatttattatggacatactgcaagtagtctaaatctt

SEQ ID NO: 12 - protein, 27 aa, Gallus gallus
MTCQTYNLFVLSVIMIYYGHTASSLNL

SEQ ID NO: 13 - DNA, 69 bp, Sus Scrofa
atgagttatacaacttatttcttagcttttcagctttgcgtgactttgtgtttttctggctcttactgc

SEQ ID NO: 14 - protein, 23 aa, Sus scrofa
MSYTTYFLAFQLCVTLCFSGSYC

SEQ ID NO: 15 - DNA, 60 bp, Influenza Virus
atgaaagtaaaactactgatcctgttatgtacatttacagctacatatgcagacacaata

SEQ ID NO: 16 - protein, 20 aa, Influenza Virus
MKVKLLILLCTFTATYADTI

SEQ ID NO: 17 - protein, 29 aa, Rattus norvegicus
MRCFISLVLGLLALEVALARNLQEHVFNS

SEQ ID NO: 18 - protein, 28 aa, Rattus norvegicus
MALHMVLVVLSLLPLLEAQNPEPANITL

SEQ ID NO: 19 - protein, 34 aa, Mus musculus
MDYYRKYAAVILVMLSMFLHILHSLPDGDFIIQG

SEQ ID NO: 20 - protein, 36 aa, Myxine glutinosa
MALSPFLAAVIPLVLLLSRAPPSADTRTTGHLCGKD

SEQ ID NO: 21 - protein, 34 aa, Lophius piscatorius
MAALWLQSFSLLVLLVVSWPGSQAVAPAQHLCGS

SEQ ID NO: 22 - protein, 34 aa, Homo Sapiens
MALWMRLLPLLALLALWGPDPAAAFVNQHLCGSH

SEQ ID NO: 23 - protein, 34 aa, Rattus norvegicus
MALWMRFLPLLALLVLWEPKPAQAFVKQHLCGPH

SEQ ID NO: 24 - protein, 34 aa, Rattus norvegicus
MALWIRFLPLLALLILWEPRPAQAFVKQHLCGSH

SEQ ID NO: 25 - protein, 25 aa, Ovis aries
MKVLILACLVALALAREQEELNVVG

SEQ ID NO: 26 - protein, 31 aa, Ovis aries
MRKSILLVVTILALTLPFLIAQEQNQEQRIC

SEQ ID NO: 27 - protein, 29 aa, Ovis aries
MMSFVSLLLVGILFWATQAEQLTKCEVFQ

SEQ ID NO: 28 - protein, 28 aa, Ovis aries
MKCLLLALGLALACGVQAIIVTQTMKGL

SEQ ID NO: 29 - protein, 25 aa, Ovis aries
  misc_feature (24)..(24): Xaa can be any naturally occurring amino acid
MKLLILTCLVAVALARPKHPIKHXG

SEQ ID NO: 30 - protein, 25 aa, Ovis aries
MKVLMKACLVAVALAKNTMEHVSSS

SEQ ID NO: 31 - protein, 26 aa, vs virus
  misc_feature (24)..(26): Xaa can be any naturally occurring amino acid
MKCLLYLAFLFIHVNCKFTIVFPXXX

SEQ ID NO: 32 - protein, 33 aa, Gallus gallus
MQYRALVIAVILLLSTTVPEVCSKSIIDRERRD

SEQ ID NO: 33 - protein, 31 aa, Apis mellifera
MKFLVNVALVFMVVYISYIYAAPEPEPAPEP

SEQ ID NO: 34 - protein, 39 aa, Rattus norvegicus
  misc_feature (31)..(32): Xaa can be any naturally occurring amino acid
MNSQVSARKAGTLLLLMMSNLLFCQNVQTLXXCXXXXCX

SEQ ID NO: 35 - protein, 35 aa, Homo Sapiens
MPGSRTSLLLAFALLCLPWLQEAGAVQTVPLSRLF

SEQ ID NO: 36 - protein, 30 aa, Homo Sapiens
MEMFQGLLLLLLLSMGGTWASKEPLRPRCR

SEQ ID NO: 37 - protein, 34 aa, Homo Sapiens
MDYYRKYAAIFLVTLSVFLHVLHSAPDVQDCPEC

SEQ ID NO: 38 - protein, 31 aa, Oryctolagus cuniculus
  misc_feature (29)..(29): Xaa can be any naturally occurring amino acid
MKLAITLALVTLALLCSPASAGICPRFAXVI

SEQ ID NO: 39 - protein, 36 aa, Rattus norvegicus
MAADSQTPWLLTFSLLCLLWPQEAGALPAMPLSSLF

SEQ ID NO: 40 - protein, 36 aa, Homo Sapiens
MATGSRTSLLLAFGLLCLPWLQEGSAFPTIPLSRLF

SEQ ID NO: 41 - protein, 37 aa, Bos taurus
MMAAGPRTSLLLAFALLCLPWTQVVGAFPAMSLSGLF

SEQ ID NO: 42 - protein, 35 aa, Bos taurus
MMSAKDMVKVMIVMLAICFLARSDGKSVKKRAVSE

SEQ ID NO: 43 - protein, 32 aa, Rattus norvegicus
MSSRLLLQLLGFWLFLSQPCRARVSEEWMDQV

SEQ ID NO: 44 - protein, 28 aa, Rattus norvegicus
MKWVTFLLLLFISGSAFSRGVFRREAHK

SEQ ID NO: 45 - protein, 28 aa, Homo Sapiens
MKWVTFISLLFLFSSAYSRGVFRRDAHK

SEQ ID NO: 46 - protein, 28 aa, Rattus norvegicus
MKWVTFLLLLFISGSAFSRGVFRREAHK

SEQ ID NO: 47 - protein, 34 aa, Gallus gallus
MRQAAAPLLPGVLLLFSILPASQQGGVPGAIPGG

SEQ ID NO: 48 - protein, 34 aa, Gallus gallus
  misc_feature (32)..(34): Xaa can be any naturally occurring amino acid
MAMAGVFVLFSFVLCGFLPDAAFGAEVDCSRXXX

SEQ ID NO: 49 - protein, 28 aa, Gallus gallus
  misc_feature (26)..(28): Xaa can be any naturally occurring amino acid
MRSLLILVLCFLPLAALGKVFGRCEXXX

SEQ ID NO: 50 - protein, 29 aa, Gallus gallus
  misc_feature (27)..(29): Xaa can be any naturally occurring amino acid
MKLILCTVLSLGIAAVCFAAPPKSVIXXX

SEQ ID NO: 51 - protein, 34 aa, Homo Sapiens
MPSSVSWGILLLAGLCCLVPVSLAEDPQGDAAQK

SEQ ID NO: 52 - protein, 33 aa, Rattus norvegicus
MSTVELSLCLLIMLAVCCYEANASQICELVAHE

SEQ ID NO: 53 - protein, 30 aa, Rattus norvegicus
MRLSLCLLTILVVCCYEANGQTLAGVCQAL

SEQ ID NO: 54 - protein, 27 aa, AD virus
MRYMILGLLALAAVCSAAKKVEFKEPA

SEQ ID NO: 55 - protein, 28 aa, Rattus norvegicus
  misc_feature (16)..(17): Xaa can be any naturally occurring amino acid
MKAAVLAVALVFLTGXXAXEFXXXDEPX

SEQ ID NO: 56 - protein, 29 aa, Rabies virus
MVPQALLFVPLLVFPLCFGKFPIYTILDK

SEQ ID NO: 57 - protein, 26 aa, Influenza virus
MKTIIALSYIFCLVFAQDLPGNDNNS

SEQ ID NO: 58 - protein, 25 aa, Influenza virus
MAIIYLILLFTAVRGDQICIGYHAN

SEQ ID NO: 59 - protein, 28 aa, Influenza virus
MNTQILVFALVAVIPTNADKICLGHHAV

SEQ ID NO: 60 - protein, 33 aa, Homo Sapiens
MALTFALLVALLVLSCKSSCSVGCDLPQTHSLG

SEQ ID NO: 61 - protein, 33 aa, Homo Sapiens
MALTFYLMVALVVLSYKSFSSLGCDLPQTHSLG

SEQ ID NO: 62 - protein, 33 aa, Homo Sapiens
MALSFSLLMAVLVLSYKSICSLGCDLPQTHSLG

SEQ ID NO: 63 - protein, 33 aa, Homo Sapiens
MASPFALLMVLVVLSCKSSCSLGCDLPETHSLG

SEQ ID NO: 64 - protein, 33 aa, Homo Sapiens; identical to SEQ ID NO: 62

SEQ ID NO: 65 - protein, 33 aa, Homo Sapiens
MALPFSLMMALVVLSCKSSCSLGCNLSQTHSLN

SEQ ID NO: 66 - protein, 33 aa, Homo Sapiens
MALPFALMMALVVLSCKSSCSLGCNLSQTHSLN

SEQ ID NO: 67 - protein, 30 aa, Homo Sapiens
MKYTSYILAFQLCIVLGSLGCYCQDPYVKE

SEQ ID NO: 68 - protein, 31 aa, Homo Sapiens
MTNKCLLQIALLLCFSTTALSMSYNLLGFLQ

SEQ ID NO: 69 - protein, 30 aa, Mus musculus
MRAPAQIFGFLLLLFPGTRCDIQMTQSPSS

SEQ ID NO: 70 - protein, 30 aa, Mus musculus
METDTLLLWVLLLWVPGSTGDIVLTQSPAS

SEQ ID NO: 71 - protein, 30 aa, Mus musculus
METDTLLLWVLLLWVPGSTGNIVLTQSPAS

SEQ ID NO: 72 - protein, 30 aa, Mus musculus
METDTLLLWVLLLWVPGADAAPTVSIFPPS

SEQ ID NO: 73 - protein, 29 aa, Mus musculus
MAWISLILSLLALSSGAISQAVVTQESAL

SEQ ID NO: 74 - protein, 29 aa, Mus musculus
MAWISLILSLLALSSGAISQAVVTQESAL

SEQ ID NO: 75 - protein, 29 aa, Mus musculus
MAWTSLILSLLALCSGASSQAVVTQESAL

SEQ ID NO: 76 - protein, 34 aa, Mus musculus
  misc_feature (28)..(34): Xaa can be any naturally occurring amino acid
MGVRMESHTRVFIFLLLWLSGTDGDIVXXXXXXX

SEQ ID NO: 77 - protein, 32 aa, Mus musculus
MDMRAPAQIFGFLLLLFPGTRCDIQMTQSPSS

SEQ ID NO: 78 - protein, 28 aa, Mus musculus
MKVLSLLYLLTAIPGIMSDVQLQESGPG

SEQ ID NO: 79 - protein, 29 aa, Mus musculus
  misc_feature (20)..(29): Xaa can be any naturally occurring amino acid
MGWSWIFLFLLSGTAGVHSXXXXXXXXXX

SEQ ID NO: 80 - protein, 29 aa, Mus musculus
  misc_feature (20)..(29): Xaa can be any naturally occurring amino acid
IKWSWISLFLLSGTAGVHSXXXXXXXXXX

SEQ ID NO: 81 - protein, 29 aa, Mus musculus
  misc_feature (20)..(29): Xaa can be any naturally occurring amino acid
MECSWVFLFLLSLTAGIHCXXXXXXXXXX

SEQ ID NO: 82 - protein, 29 aa, Mus musculus
  misc_feature (20)..(29): Xaa can be any naturally occurring amino acid
MEWSGVFIFLLSVTAGVYSXXXXXXXXXX

SEQ ID NO: 83 - protein, 29 aa, Mus musculus
MGWSFIFLFLLSVTAGVHSEVQLQQSGAE

SEQ ID NO: 84 - protein, 25 aa, Canis lupus
  misc_feature (1)..(2): Xaa can be any naturally occurring amino acid
XXPLLILAFLXAAVATPTDDDDKIV

SEQ ID NO: 85 - protein, 25 aa, Canis lupus
  misc_feature (3)..(3): Xaa can be any naturally occurring amino acid
ALXIIFLALLXAXVAFPIDDDDKIV

SEQ ID NO: 86 - protein, 27 aa, Canis lupus
  misc_feature (7)..(7): Xaa can be any naturally occurring amino acid
AFLILVXAFALXXVAFXXXVPAIXPVX

SEQ ID NO: 87 - protein, 26 aa, Canis lupus
  misc_feature (1)..(2): Xaa can be any naturally occurring amino acid
XXLILVFGALLXAIYXQXAFVXXXXX

SEQ ID NO: 88 - protein, 25 aa, Canis lupus
  misc_feature (1)..(2): Xaa can be any naturally occurring amino acid
XXFFLLLXVIGFXVAQYAPHXXXXX

SEQ ID NO: 89 - protein, 25 aa, Mus musculus
MKFFLLLSLIGFCWAQYDPHTQYGR

SEQ ID NO: 90 - protein, 25 aa, Mus musculus
MKFVLLLSLIGFCWAQYDPHTSDGR

SEQ ID NO: 91 - protein, 20 aa, Rattus norvegicus
LLSLIGFCYAQYDPHTADGR

SEQ ID NO: 92 - protein, 29 aa, Oryctolagus cuniculus
MMPLVPLLLVSIVFPGIQATQLTRCELTE

SEQ ID NO: 93 - protein, 29 aa, Sus sofra
MMSFVSLLVVGILFPAIQAKQFTKCELSQ

SEQ ID NO: 94 - protein, 26 aa, Rattus norvegicus
MKRLLILSLLLEAVCGNENFVGHQVL

SEQ ID NO: 95 - protein, 36 aa, Bos taurus
MPRLCSSRSGALLLALLLQASMEVRGWCLESSQCQD

SEQ ID NO: 96 - protein, 30 aa, Sus sofra
MAWQGLLLAACLLVLPSTMADCLSGCSLCA

SEQ ID NO: 97 - protein, 30 aa, Homo Sapiens
MARFLTLCTWLLLLGPGLLATVRAECSQDC

SEQ ID NO: 98 - protein, 31 aa, Sus sofra
  misc_feature (30)..(31): Xaa can be any naturally occurring amino acid
MQRLCAYVLIHVLALAACSEASWKPGFQLXX

SEQ ID NO: 99 - protein, 31 aa, Mus musculus
MDRRRMPLWALLLLWSPCTFSLPTGTTFERI

SEQ ID NO: 100 - protein, 31 aa, Trypanosome
MVKAIASLMLLHIWAIEEIKAERQAPSVSRT

SEQ ID NO: 101 - protein, 33 aa, Ictalurus punctatus
MSSSPLRLALALMCLVSAVGVISCGRPHVVLNS

SEQ ID NO: 102 - protein, 36 aa, Lophius piscatorius
MVSSSRLRCLLVLLLSLTVSISCSFAGQRDSKLRLL

SEQ ID NO: 103 - protein, 38 aa, Lophius piscatorius
MKMVSSSRLRCLLVLLLSLTASISCSFAGQRDSKLRLL

SEQ ID NO: 104 - protein, 33 aa, Lophius piscatorius
MQCIRCPAILALLALVLCGPSVSSQLDREQSDN

SEQ ID NO: 105 - protein, 32 aa, Lophius piscatorius
MGFLKFSPFLVVSILLLYQACGLQAVPLRSTL

SEQ ID NO: 106 - protein, 31 aa, Lophius piscatorius
MKRIHSLAGILLVLGLIQSSCRVLMQEADPS